(19) United States
(12) Patent Application Publication
(10) Pub. No.: US 2014/0112985 A1
(43) Pub. Date: Apr. 24, 2014

(54) METHOD OF PREVENTION AND TREATMENT OF CLOSTRIDIUM DIFFICILE INFECTION

(71) Applicant: POLONEZ THERAPEUTICS LLC, Newtown, PA (US)

(72) Inventors: Wieslaw J. Bochenek, Newtown, PA (US); Michael L. Corrado, Perkasie, PA (US)

(73) Assignee: POLONEZ THERAPEUTICS LLC, Newtown, PA (US)

(21) Appl. No.: 13/657,124

(22) Filed: Oct. 22, 2012

(51) Int. Cl. A61K 35/74; A61K 9/48; A61K 9/52; A61K 9/20; A61J 1/00; A61K 36/064; A61K 36/06; A61P 1/00; A61P 1/14; A61K 9/50; A61K 9/00

(52) U.S. Cl. 424/458; 424/490; 424/451; 424/464; 424/468; 424/400; 424/93.51; 424/93.4; 424/93.41; 424/93.45; 424/93.48; 206/438

(57) ABSTRACT

This invention relates to prophylactic and/or therapeutic application of microorganism species that are, for example, administered orally as delayed release formulation designed to release its microbial content to the distal small intestine and/or colon in high quantities and density, which is a “normalized” approach to repopulate the colonic flora as a method of prevention and/or treatment of, for example, Clostridium difficile colitis.

### BACKGROUND OF THE INVENTION

1. Field of Invention

This invention relates to prophylactic and/or therapeutic application of microorganism species that are, for example, administered orally as delayed release formulation designed to release its microbial content in the distal small intestine, just proximal to the colon, or in the colon in high quantities and density, which is a “normalized” approach to repopulate the colonic flora as a method of prevention and/or treatment of, for example, Clostridium difficile colitis.

2. Background of Invention

Clostridium difficile is a toxin forming bacterium that may cause inflammation of the colonic mucosa also known as pseudomembranaceous colitis. The most prominent symptom of the disease is diarrhea that is often referred to as Clostridium difficile Associated Diarrhea (CDAD). The disease most often develops in patients who have been treated with antibiotics and thus the often used term is Antibiotic Associate Diarrhea (AAD) with Clostridium difficile as a frequent cause of it. Infection caused by Clostridium difficile became recently a significant threat due to emergence of bacterial strain NAP01 that is capable of producing large quantities of toxin leading to inflammation of the colon that frequently affects debilitated hospitalized patients who are at particularly high risk. The recurrence of infection after seemingly successful treatment is the cause of increased mortality and significantly increases the cost of treatment.

Clostridium difficile is an obligate anaerobe which is spore forming. It is relatively resistant to most antibacterials in use, which antibacterials, when given to individuals, kill off more susceptible bacteria leaving the gut ecologic niche available to Clostridium difficile to re-populate and overgrow the colon. Bacteria, including Clostridium difficile are believed to “cross talk” by the elaboration of certain chemicals which can up-regulate genes. These genes then, in their “switched on state”, produce certain substances which give the organism an ability to exploit their environment. It is thought that these substances that allow “cross talking” between bacterial cells to turn on these genes occurs when specific densities of bacteria are encountered. This required density effect is referred to a “quorum sensing”(1).

It is the ability of “normal bacterial flora”, or the bacteria that normally populate healthy gastrointestinal tract, and particularly colon, referred to as intestinal microbiome, to resist and prevent this overgrowth of a single species that is essential to normal colonic health. While toxigenic Clostridium difficile can be a normal component of one's bowel flora, its presence in low numbers precludes the ability to reach a critical number for quorum sensing to occur and thus is not able to cause toxin related colitis with resulting diarrhea.

However, once the normal flora is disrupted by antibiotics, Clostridium difficile which is resistant to the majority of antibiotics has less competition for this ecologic niche and replicates (2). Thus, the disruption of the microbiome in the colon creates environment conducive to overgrowth of pathogenic Clostridium difficile resulting in significant health hazard.

All references cited herein are incorporated herein by reference in their entireties.

### BRIEF SUMMARY OF THE INVENTION

This invention uses a “normalized” approach to repopulate the colonic flora as a method of treatment of, for example, Clostridium difficile colitis. This method uses two important principles that will allow for efficacious achievement of this goal:

a) protection of the donated microorganisms from the deleterious effects of the environment of the upper gastrointestinal tract, specifically protection from gastric acid and highly digestive contents of the duodenum and upper small intestine and prevention of the dilution and decline in their number during transit through the small bowel and before reaching the colon; and

b) increase of quantity and density of beneficial microorganisms in an attempt to repopulate the colon with microbial species that are typical of healthy colonic echo-system and/or the organisms known to be antagonistic to Clostridium difficile in order to suppress the toxin producing Clostridium difficile species.

It is believed that this approach recapitulates the suppressive effects of healthy bowel flora against the overgrowth of pathogenic Clostridium difficile. This approach has been shown effective in treating severe Clostridium difficile infections using the so called fecal transplants (3,4,5). Fecal transplant despite being highly efficacious has not gained popularity because it is technically difficult.

This invention pertains to prophylactic and/or therapeutic application of microorganism species that are, for example, administered orally as delayed release formulation designed to release its microbial content just proximal to the colon or in the colon in high quantities and density. Such applications are to be used easily and repeatedly daily and over extended periods of time. Microorganisms in this invention can be formulated as capsules, tablets, caplets or other specialty formulated oral delivery systems that will release their content in the distal small intestine and/or colon. Alternatively, the microorganisms may be administered directly to those locations as suspension via catheter or cannula if such access is readily available. The microorganisms in this invention are representative of healthy colonic bacterial flora primarily anaerobic bacteria in its vegetative or spore forms as well as some aerobic bacteria and other microorganisms that will promote development of healthy colonic bacterial flora and/or are known to be antagonistic and capable of suppressing growth of Clostridium difficile such as Saccharomyces boulardii (6,7).

It is imperative that the formulation used for the delivery of microorganisms will protect their viability during storage and until their release in the distal small intestine and/or colon.

The invention provides a method of treatment or prevention of Clostridium difficile infection in a patient in need of such treatment or prevention by administration of a probiotic delivery system for administering a plurality of live probiotic microorganisms to the intestine of an individual in need thereof, said probiotic delivery system comprising: a probiotic agent, wherein said probiotic agent comprises at least one species of live probiotic microorganisms; optionally, at least one additional agent selected from the group consisting of antibiotics, bismuth containing compounds, intestinal motility agents, and combinations thereof; and a delivery vehicle, wherein the oral delivery vehicle releases the probiotic agent to at least the distal small intestine and/or colon of the intestine of the individual. The invention further provides a method wherein the treatment can be used as an adjunct to antibiotic treatment of Clostridium difficile and antibiotic associated diarrhea. The invention further provides a method wherein the treatment is prophylaxis to prevent initial and recurrent or refractory Clostridium difficile infection. The invention further provides a method wherein the treatment is prophylactic use applied in patients at risk of Clostridium difficile infection that include antibiotic use, advanced age, co-morbidities, underlying disease severity, and exposure to other patients with Clostridium difficile infection, prolonged hospitalization. The invention provides a method of treating a disease or condition in a patient by using a personalized probiotic delivery system according to the invention, wherein the means for containing said therapeutic dosage or dosages is selected from the group consisting of: the first and optional unit dosages are packaged together in a single package or packette; the first and optional unit dosages are packaged separately in a plurality of packages or packettes; a blister packet; a lidded blister; or blister card or packets; a shrink wrap, and with both drugs released upon opening of the single package or packette; a plurality of packages or packettes; blister packet; lidded blister or blister card or packets; or shrink wrap; a blister pack; a container; and a device.

The invention further provides a method of treating a disease or condition in a patient by using a personalized probiotic delivery system according to the invention, wherein said system is selected from the group consisting of a blister package comprising: a) a rupturable substrate b) a layer forming one or more blisters over the rupturable substrate, wherein each of the one or more blisters contain one or more unit dosage forms. The invention provides a method of treatment or prevention of Clostridium difficile infection in a patient in need of such treatment or prevention by administration of a probiotic formula directly to the distal small intestine and/or colon of a patient as a suspension using a route of administration selected from the group consisting of a catheter and a cannula. The invention further provides a method wherein the probiotic formula is in the form of coated granules. The invention further provides a method wherein the coated granules have an immediate release coating, or a delayed release coating, or are combination of immediate and delayed release granules. The invention further provides a method wherein the coated granules are mixed as a suspension and administered orally. The invention further provides a method wherein the probiotic formula is in a dosage form selected from the group consisting of a prefilled syringe, a user filled syringe, sachet, large capsule, and a squeezable tube that will allow administration of the probiotic microorganisms through a tube or catheter, further wherein the administration is through a stoma or administration rectally. The invention further provides a method wherein the dosage form protects the viability of the probiotic agent during storage and until their release in the distal small intestine and/or colon. The invention further provides a method wherein said probiotic delivery system is administered with a regimen selected from the group consisting of once a day and multiple times a day, with food, without food, and combinations thereof.

The invention provides an oral probiotic delivery system for administering live probiotic microorganisms to an individual in need thereof, said probiotic delivery system comprising: a probiotic agent, wherein said probiotic agent comprises at least one species of live probiotic microorganisms; optionally, at least one additional agent selected from the group consisting of antibiotics, bismuth containing compounds, intestinal motility agents, and combinations thereof; and a delivery vehicle, wherein the oral delivery vehicle releases the probiotic agent to the distal small intestine and/or colon of the intestine of the individual. The invention further provides an oral probiotic delivery system wherein the delivery vehicle is selected from the group consisting of pills, tablets, caplets, capsules, soft gels, and coated probiotic granules, that will release the probiotic agent in the distal small intestine and/or colon. The invention further provides an oral probiotic delivery system wherein the additional agent is present, and is in a dosage form selected from immediate release, delayed release, extended release which is released in the distal small intestine and/or colon, and targeted release which is targeted to be released in the distal small intestine and/or colon. The invention further provides an oral probiotic delivery system wherein the additional agent is targeted to release the additional agent in the distal small intestine and/or colon of the intestine of the individual. The invention further provides an oral probiotic delivery system wherein the coated probiotic granules are in a sachet. The invention further provides an oral probiotic delivery system wherein the coated granules are selected from the group consisting of probiotic granules with an immediate release coating, probiotic granules with a delayed release coating, microencapsulated probiotic granules, and combinations thereof. The invention further provides an oral probiotic delivery system wherein the coated granules are mixed as a suspension and administered orally. The invention further provides an oral probiotic delivery system wherein the coated granules can be applied directly to food as a sprinkle. The invention further provides an oral probiotic delivery system wherein the coated granules are mixed as a suspension and administered directly to the intestine, optionally through a catheter or cannula.

The invention further provides an oral probiotic delivery system wherein the coated probiotic granules are in a capsule, which optionally has a coating selected from immediate release, delayed release, extended release which is released in the distal small intestine and/or colon, and targeted release which is targeted to be released in the distal small intestine and/or colon. The invention further provides an oral probiotic delivery system wherein the coated probiotic granules are in a tablet or caplet, which optionally has a coating selected from immediate release, delayed release, extended release which is released in the distal small intestine and/or colon, and targeted release which is targeted to be released in the distal small intestine and/or colon. The invention further provides an oral probiotic delivery system wherein the probiotic agent is primarily anaerobic bacteria, wherein the anaerobic bacteria is in vegetative, spore, or a mixture of vegetative and spore forms, further wherein the probiotic agent will promote development of healthy colonic bacterial flora. The invention further provides an oral probiotic delivery system wherein the probiotic agent is capable of suppressing growth of C. diff. The invention further provides an oral probiotic delivery system wherein the probiotic agent is primarily aerobic bacteria, wherein the anaerobic bacteria is in vegetative, spore, or a mixture of vegetative and spore forms, further wherein the probiotic agent will promote development of healthy colonic bacterial flora. The invention further provides an oral probiotic delivery system wherein the delivery vehicle protects the viability of the probiotic agent during storage and until their release in the distal small intestine and/or colon. The invention further provides an oral probiotic delivery system, wherein the delivery vehicle is coated such that the probiotic agent is released in the distal small intestine and/or colon. The invention further provides an oral probiotic delivery system wherein the delivery vehicle will pass intact through the stomach, the duodenum and the upper small intestine to release the probiotic agent in the distal small intestine and/or the colon.

The invention further provides an oral probiotic delivery system wherein the delivery vehicle is coated such that will ensure the delayed release of its content and will offer required shelf life stability. The invention further provides an oral probiotic delivery system wherein the delivery vehicle is a capsule, wherein the capsule comprises materials selected from the group consisting of hydroxy-propyl-methyl-cellulose, gelatin, starch, and combinations thereof. The invention further provides an oral probiotic delivery system wherein the delivery vehicle has a pH dependent coating. The invention further provides an oral probiotic delivery system wherein the delivery vehicle is coated with a copolymer selected form the group consisting of methacrylic acid, methacrylates, and combinations thereof that dissolve at pH 5.5 to 7.0, and may be used to achieve distal small intestinal and/or colonic delivery. The invention further provides an oral probiotic delivery system wherein the thickness of the coating constitutes additional factor that can be employed to adjust time required for disintegration of the coated formulation delivery vehicle. The invention further provides an oral probiotic delivery system wherein the delivery vehicle is pre-coated prior to filling with the probiotic agent to reduce damaging effect of the formulation process on the probiotic agent, and will ensure tight and stable closure of delivery vehicle. The invention further provides an oral probiotic delivery system wherein the delivery vehicle achieves distal small intestinal and/or colonic delivery by dissolution that is time dependent. The invention further provides an oral probiotic delivery system wherein the delivery vehicle has delayed delivery using hydrogel plug. The invention further provides an oral probiotic delivery system wherein the delivery vehicle time dependent delivery systems utilize ethylcellulose as the release determining polymer. The invention further provides an oral probiotic delivery system wherein the delivery vehicle has delayed delivery based on a combination of pH dependent and time dependent systems. The invention further provides an oral probiotic delivery system wherein the delivery vehicle has delayed delivery based on bacterial degradation of the formulation coating, such as degradation of polymers containing azo bonds that are cleaved by bacterial azo reductase enzyme. The invention further provides an oral probiotic delivery system wherein the probiotic agent is selected from the group consisting of Streptococcus, Enterococcus, Staphylococcus, Micrococcus, Leuconostoc, Pediococcus, Stomatococcus, Corynebacterium, Arthrobacter, Brevibacterium, Rothia, Arcanobacterium, Aureobacterium, Microbacterium, Gardnerella, Kurthia, Bacillus, Escherichia, Enterobacter, Ewingella, Hafnia, Klebsiella, Kluyvera, Leciercia, Leminorella, Moellerella, Obesumbacterium, Pragia, Pantoea, Photorhabdus, Proteus, Providencia, Rahnella, Serratia, Tatumella, Citrobacter, Clostridium, Peptostreptococcus, Propionibacterium, Lactobacillus, Eubacterium, Bifidobacterium, Mobiluncus, Bacteroides, Porphyromonas, Prevotella, Fusobacterium, Sutterella, Bilophila, Butyrrivibrio, Catonella, Dialister, Johnsonella, Saccharomyces, Pichia, Faecalibacterium, Butyricoccus, and combinations thereof. The invention further provides an oral probiotic delivery system wherein the probiotic agent is Saccharomyces boulardi. The invention further provides an oral probiotic delivery system wherein the probiotic agent is in vegetative or spore form. The invention further provides an oral probiotic delivery system wherein the probiotic agent is intestinal or fecal contents cultured from donors selected from the patient, patient relatives, and unrelated human donors.

The invention provides a personalized probiotic delivery kit for administering live probiotic microorganisms to selected regions of the intestine of an individual in need thereof, said personalized probiotic delivery kit comprising: a first probiotic delivery system, which comprises a probiotic agent, wherein said probiotic agent comprises at least one species of live probiotic microorganisms; and a delivery vehicle, wherein the oral delivery vehicle releases the probiotic agent to the distal small intestine and/or colon of the intestine of the individual; optionally, the kit comprises additional probiotic delivery systems, each of which comprises a probiotic agent, wherein said probiotic agent comprises at least one species of live probiotic microorganisms; and a delivery vehicle wherein the oral delivery vehicle releases the probiotic agent to at least one selected region of the intestine of the individual; and optionally, at least one additional agent selected from the group consisting of antibiotics, bismuth containing compounds, intestinal motility agents, and combinations thereof, wherein the number and kinds of probiotic microorganisms in the optional additional probiotic delivery systems are designed to vary the number of probiotic microorganisms administered may be increased or decreased and the species of the probiotic microorganisms may differ depending whether it is prophylactic or therapeutic use and ranging from 1-25 billion of cells per administered unit which can be modified over time in the Clostridium difficile treatment phase and the prevention phase. The invention further provides a personalized probiotic delivery kit wherein said probiotic agent is in a delivery vehicle, wherein said delivery vehicle is selected from the group consisting of pills, tablets, caplets, capsules, and soft gels, and coated probiotic granules. The invention further provides a personalized probiotic delivery kit wherein the delivery vehicle is in a dosage form selected from immediate release, delayed release, extended release, and targeted release. The invention further provides a personalized probiotic delivery kit wherein the coated probiotic granules are in a sachet. The invention further provides a personalized probiotic delivery kit wherein said probiotic agent, said optional additional probiotic agents, and said optional additional agents are in separate unit dosages. The invention further provides a personalized probiotic delivery kit wherein said probiotic agent, and said optional additional agents are in the same unit dosage form. The invention further provides a personalized probiotic delivery kit wherein said optional additional probiotic agents are in separate unit dosages. The invention further provides a personalized probiotic delivery kit wherein said probiotic agent, said optional additional probiotic agents, and said optional additional agents are in the same unit dosage form.

The invention further provides a personalized probiotic delivery kit wherein said system comprises unit dosages packaged in packages selected from the group consisting of: a blister package comprising: a) a rupturable substrate b) a layer forming one or more blisters over the rupturable substrate, wherein each of the one or more blisters contain one or more unit dosage forms. The invention further provides a personalized probiotic delivery kit wherein said system containing said dosage or dosages is selected from the group consisting of: the first and optional unit dosages are packaged together in a single package or packette; the first and optional unit dosages are packaged separately in a plurality of packages or packettes; a blister packet; a lidded blister; or blister card or packets; a shrink wrap, and with both drugs released upon opening of the single package or packette; a plurality of packages or packettes; blister packet; lidded blister or blister card or packets; or shrink wrap; a blister pack; a container; and a device, and further wherein the dosages are separated from each other within the personalized pharmaceutical packaging system. The invention further provides a personalized probiotic delivery kit wherein the probiotic agent is anaerobic microorganisms, manufactured and stored in atmosphere devoid of oxygen. The invention further provides a personalized probiotic delivery kit wherein the probiotic agent is anaerobic microorganisms, and the delivery vehicle is packaged in an atmosphere containing predominantly nitrogen, helium, or other inert gas, that will not adversely affect anaerobic bacterial contents. The invention further provides a personalized probiotic delivery kit wherein the package will not be permeable to air and oxygen if anaerobic species is used and protect from humidity and exposure to light during storage to ensure required shelf storage stability of the formulation. The invention further provides a personalized probiotic delivery kit wherein probiotic microorganisms used in this invention can come either from commercial suppliers or can be custom cultured using stock microorganisms or using human feces or intestinal contents as the starting material.

### DETAILED DESCRIPTION OF THE INVENTION

### Definition of Terms and Conventions Used

Terms not specifically defined herein should be given the meanings that would be given to them by one of skill in the art in light of the disclosure and the context. As used in the specification and appended claims, however, unless specified to the contrary, the following terms have the meaning indicated and the following conventions are adhered to:

Aerobic bacteria: bacteria that require oxygen for growth or that can grow in presence of oxygen.

Anaerobic bacteria: Bacteria that do not require oxygen for growth and may even be harmed by oxygen

Antibiotic Associated Diarrhea (AAD): Diarrhea that is related to treatment with antibiotics and often caused by proliferation with toxin producing Clostridium difficile.

Probiotics: According to FAO/WHO, probiotics are: “Live microorganisms which when administered in adequate amounts confer a health benefit on the host” (Report of a Joint FAO/WHO Expert Consultation on Evaluation of Health and Nutritional Properties of Probiotics in Food Including Powder Milk with Live Lactic Acid Bacteria (October 2001). “Health and Nutritional Properties of Probiotics in Food including Powder Milk with Live Lactic Acid Bacteria”. Food and Agriculture Organization of the United Nations, World Health Organization. www.who.int/foodsafety/publications/fs_management/en/probiotics.pdf. Retrieved 2009-11-04.)

Clostridium difficile: anaerobic, spore forming Gram positive bacterium, part of microbiome echosystem of the human colon. Pathogenic species produce toxin A and B that are the cause of Clostridium difficile colitis.

Clostridium difficile associated diarrhea (CDAD): diarrhea that is the main symptom of toxin producing Clostridium difficile infection.

Fecal transplant: Method of transplanting processed fecal material containing fecal flora obtained from a healthy donor into a recipient as a therapeutic means to repopulate patient's colon with healthy bacterial flora. Such transplants can be administered using per rectum route and also can be administered endoscopically orally

Spore: is a dormant and non-reproductive structure produced by certain bacteria, e.g. Clostridium difficile. Spores do not require nutrition and are capable of survival in conditions where the vegetative form of the bacterium could not exist. They can reactivate into the vegetative form when environmental conditions become favorable.

Healthy colonic bacterial flora: Complex microbiome ecosystem existing in the colon, composed primarily of anaerobic bacteria.

HPMC: Hydroxypropyl methyl cellulose is a semisynthetic plant origin material that can be used to make capsules.

Pseudomembranous colitis: Inflammation of the colonic mucosa most frequently associated with toxin producing Clostridium difficile infection.

Quorum sensing: system of stimulus and response correlated to population density. Many species of bacteria use quorum sensing to coordinate gene expression according to the density of their local population.

The present invention relates to the method of use of probiotic microorganisms as therapeutic and/or preventative means of combating or preventing Clostridium difficile infection or recurrent infection. In health, Clostridium difficile resides primarily in the colon as part of a complex microbial colonic echo system remaining in commensal relationship with the human host. However, some strains of Clostridium difficile possess gene for production of powerful toxins (Toxin A and B) that may exert damaging effect on the colon mucosa. Clostridium difficile toxins can be found in the stool of 15%-25% of patients with antibiotic associated diarrhea and more than 95% of patients with pseudomembranous colitis (8). Clostridium difficile associated diarrhea (CDAD) can range from uncomplicated diarrhea to sepsis and even death. In health, such toxigenic Clostridium difficile species either do not exist or their quantities are too small to cause disease. This may change, however, when the microbiome echo balance of the colon is disrupted. This may occur as result of treatment with broad spectrum antibiotics for any infection. Many non-pathogenic microbial species are then eliminated leaving the colonic microbial niche open to proliferation of Clostridium difficile and the quorum sensing effect will aid in dissemination of toxin producing gene and the development of Clostridium difficile associated diarrhea and colitis.

This invention relates to the method of administration of probiotic organisms using a delivery formulation that will pass intact through the stomach, the duodenum and the upper small intestine to release the probiotic microorganisms in either the distal small intestine and/or the colon. The benefits from the delayed release of the probiotic content are, for example, that it will not be harmed by the acidic content of the stomach and the highly digestive milieu of the duodenum and will not be subject to dilution and competition with the indigenous bacterial flora in the upper segments of the small intestine. Such probiotic organisms introduced to the lower parts of the gastrointestinal tract with minimal loss of their quantity and at higher densities will have greater chance of survival and growth and be able to repopulate or aid in the repopulation of the disease affected colon with the normal bacterial flora.

The utility of this invention relies on, for example, the competition between probiotic organisms competing with pathogenic bacteria for the same ecologic niche thereby which niche can only support a certain total bacterial population, hence limiting the pathogenic population to below the level required for quorum sensing. It is important that probiotic organisms have to reach the area of the colon affected by infection in the best viable condition and quantities to exert its effect. Thus, the use of delayed release formulation for the delivery of probiotic organisms will preserve their viability and prevent probiotic organism count loss and their dilution by the intestinal contents that would be the case if the formulation were to release its content in the proximal intestine.

The microbial species that are used in this invention may be, for example, a single species or combination of species of vegetative forms and/or, when applicable, spore forms of all species of, for example, the following genera: Streptococcus, Enterococcus, Staphylococcus, Micrococcus, Leuconostoc, Pediococcus, Stomatococcus, Corynebacterium, Arthrobacter, Brevibacterium, Rothia, Arcanobacterium, Aureobacterium, Microbacterium, Gardnerella, Kurthia, Bacillus, Escherichia, Enterobacter, Ewingella, Hafnia, Klebsiella, Kluyvera, Leciercia, Leminorella, Moellerella, Obesumbacterium, Pragia, Pantoea, Photorhabdus, Proteus, Providencia, Rahnella, Serratia, Tatumella, Citrobacter, Clostridium, Peptostreptococcus, Propionibacterium, Lactobacillus, Eubacterium, Bifidobacterium, Mobiluncus, Bacteroides, Porphyromonas, Prevotella, Fusobacterium, Sutterella, Bilophila, Butyrrivibrio, Catonella, Dialister, Johnsonella, Saccharomyces, Pichia, Faecalibacterium, Butyricoccus.

The bacterial species that are particularly affected in the course of Clostridium difficile infection are the indigenous to colon mainly anaerobic but also aerobic species (9, 10). Fecal microbiome imbalance is typically found in patients with recurrent Clostridium difficile infection after successful treatment of the initial episode. Re-establishment of the normal bacterial microbiome, mainly anaerobic bacteria, has been shown to benefit such patients. The most successful method used for the reestablishment of normal colonic microbial flora is fecal transplantation. This treatment modality has been the subject of recent reviews (11,12,13). Numerous techniques of fecal transplant are used and this includes rectal instillation of fecal material from healthy donors as rectal enema, or through a colonoscope instillation and also using peroral instillation using gastroscope or nasogastric tube. Successful results have also been published using cultured fecal anaerobic bacteria instead of infusing fecal matter per se (14). It thus appears that the success of the fecal transplant method is related to the reestablishment of normal colonic bacterial flora and particularly non-pathogenic anaerobic bacterial species. While treatment using fecal transplant is highly effective, such treatment is logistically difficult requiring specialized laboratory facilities and personnel trained in collection, processing and storing of fecal specimens and also the availability of healthy donors. Moreover, the procedure may carry risks and is associated with ethical constraints.

The present invention relates to the use of certain bacterial species that are typical of normal colon microbiome that can be cultured in the laboratory on technical scale, stored safely for extended periods of time and used when needed. Such bacteria could be applied orally as capsule, tablet, caplet or any other delayed release formulation capable of delivering intact organisms to the distal gastrointestinal tract. Distal gastrointestinal delivery to the area of distal ileum and/or colon is important in protecting administered microorganisms from gastric acid and harmful effects of the contents of the upper gastrointestinal secretions and also in preventing their dilution and their loss due to competition with indigenous small intestinal bacterial flora. Delivery of the administered probiotic microorganisms to the area of the distal ileum and/or colon will ensure their introduction to the environment where existing microbiome imbalance favors uninhibited growth of Clostridium difficile species. Oral use of such formulations containing therapeutic organisms, primarily anaerobic species, has an advantages over the previous methods of fecal transplant in that it is easy to administer, does not require laboratory facilities nor the availability of healthy donors, is inexpensive and can be used repeatedly in the course of the day and also daily for prolonged periods of time. Therefore, such treatment can be used as an adjunct to standard antibiotic treatment of Clostridium difficile and antibiotic associated diarrhea. It also can be used after successful treatment to prevent recurrent Clostridium difficile infection that is reported in up to 30% of successfully treated initial infections (15). Fecal transplant has been shown as an effective treatment of recurrent and refractory Clostridium difficile infection (16). The method of treatment in this invention could also be used as prophylaxis to prevent initial and recurrent or refractory Clostridium difficile infection. Prophylactic use could be applied in patients at risk of Clostridium difficile infection that include antibiotic use, advanced age, co-morbidities, underlying disease severity, prolonged hospitalization, and exposure to other patients with Clostridium difficile infection.

The formulation relative to the present invention is such that the probiotic organisms are protected from harm in the manufacturing process and thus will maintain their viability. The most suitable formulation is the specialty coated capsule, but also could be caplet, tablet, or other suitable form that will ensure delayed release of the probiotic microorganisms in the ileum and/or colon. The invention provides that the probiotic organisms used will not be subject to harmful conditions in the manufacturing process and also their viability will be supported by a safe packaging and storage of the formulation. Such physical conditions during the storage of unformulated and formulated probiotic material and during the manufacturing process that may harm probiotic microorganisms include and are not limited to temperature, pressure applied during the formulation process, vapors, humidity, pH, exposure to light, radiation, and exposure to oxygen in the air, or otherwise, if the probiotic microorganisms belong to the category of anaerobes.

The formulation used for administration of probiotic organisms may be specialty coated that meets the requirements of the present invention, i.e. the content has to be released in the distal small intestine and/or colon. A preferred formulation is a capsule that offers the least damaging effect to the microbial content in the formulation process as compared with other formulations. The material used for manufacturing of capsules is such that it is amenable to coating that will ensure the delayed release of its content and will offer extended shelf storage stability. A preferred material used for manufacturing of capsules that will meet the requirements of the present invention is hydroxy-propyl-methyl-cellulose (U.S. Pat. No. 7,094,425), although other materials may be used as well such as gelatin, starch, and other. Specialty coating for such delayed release formulation may be achieved using different principles. The most convenient is the pH dependent coating. A review of pH based delivery coating systems has been recently published (17). Thus, coating utilizing copolymer consisting of methacrylic acid and methacrylates that preferably dissolve at pH 5.5 to 7.0 may be used to achieve distal small intestinal and colonic delivery. The thickness of the coating constitutes additional factor that can be employed to adjust time required for disintegration of the coated formulation.

Examples of such coated formulations have been published (18). Thus, HPMC capsules coated with polymer Eudragit® FS30D released its content in the distal small intestine and primarily in the proximal colon. Another example is coating utilizing Eudragit L100 and Eudragit S100 at 80:20 ratio. Starch seamless capsules such coated have achieved distal ileal and colonic delivery (19 and U.S. Pat. No. 6,228,396 B1). Pre-coating of capsules for delayed release prior to filling with probiotic microorganisms may be the preferred method that will further reduce damaging effect of the formulation process on the microorganism content and will ensure tight and stable closure of capsules (20). Some other principles for achieving distal small intestinal and/or colonic delivery may utilize dissolution that is time dependent. Examples of such delayed delivery are formulations using hydrogel plug such as in PULSINCAP™ (WO 90/09168). Other time dependent delivery systems utilize ethylcellulose as the release determining polymer (21, 22). Other examples are the TIMECLOCK® system (23) and the Time-Controlled Explosion (TES) system (24). Also a combination of pH dependent and time dependent systems can be applied (25). Some of the colonic targeted delivery systems are based on bacterial degradation of the formulation coating. Example of this is degradation of polymers containing azo bonds that are cleaved by colonic bacteria azo reductase enzyme. Various polymers of such type have been described (26,27,28). Other than azo based polymers can also be utilized to achieve colonic delivery (29,30,31, U.S. Pat. No. 6,368,629 B1).

The method of prevention of and treatment of Clostridium difficile infection described in this invention include:

### A. Prophylatic Use:

In this embodiment, repeated daily, or scheduled otherwise use of delayed release formulation of probiotic microorganisms is intended to deliver high quantities of probiotic microorganisms to the distal small intestine and/or colon, the site of colonization by the toxigenic species of Clostridium difficile. Such applications can be initiated in individuals at risk of Clostridium difficile infection at the time of relative health, at the time of anticipated treatment with antibiotics, or during treatment with antibiotics that may be conducive to the development of Clostridium difficile associated diarrhea. Prophylactic treatment can also be used at the time of treatment of Clostridium difficile infection, as a measure to prevent recurrent infection, or can be initiated at any time after treatment of Clostridium difficile infection with antibacterial agent has been completed.

### B. Therapeutic Use:

In this embodiment, daily repeated, or scheduled otherwise administration of delayed release formulation of probiotic microorganisms is used after the development of symptoms suggestive of Clostridium difficile infection, or after such infection has been confirmed by diagnostic tests. Delayed release probiotic microorganisms can be used alone or as an adjunctive treatment to antibiotic treatment or other treatment of Clostridium difficile infection. Such probiotic use is intended to shorten the duration of Clostridium difficile infection and lessen the severity of diarrhea and to lessen the recurrent Clostridium difficile infection.

### Dosage Forms

The compositions of the present invention can provided in the form of a minicapsule, a capsule, a tablet, an implant, a troche, a lozenge (minitablet), a temporary or permanent suspension, an ovule, a suppository, a wafer, a chewable tablet, a quick or fast dissolving tablet, an effervescent tablet, a granule, a film, a sprinkle, a pellet, a bead, a pill, a powder, a triturate, a platelet, a strip or a sachet. Compositions can also be administered after being mixed with, for example yoghurt or fruit juice and swallowed or followed with a drink or beverage. These forms are well known in the art and are packaged appropriately. The compositions can be formulated for oral or rectal delivery.

The compositions of the invention can be coated with one or more enteric coatings, seal coatings, film coatings, barrier coatings, compress coatings, fast disintegrating coatings, or enzyme degradable coatings. Multiple coatings can be applied for desired performance. Further, the dosage form can be designed for immediate release, pulsatile release, controlled release, extended release, delayed release, targeted release, synchronized release, or targeted delayed release. For release control, solid carriers can be made of various component types and levels or thicknesses of coats, with or without an active ingredient. Such diverse solid carriers can be blended in a dosage form to achieve a desired performance. The definitions of these terms are known to those skilled in the art. In addition, the dosage form release profile can be affected by a polymeric matrix composition, a coated matrix composition, a multiparticulate composition, a coated multiparticulate composition, an ion-exchange resin-based composition, an osmosis-based composition, or a biodegradable polymeric composition. Without wishing to be bound by theory, it is believed that the release may be effected through favorable diffusion, dissolution, erosion, ion-exchange, osmosis or combinations thereof.

When formulated as a capsule, the capsule can be a hard or soft gelatin capsule, a starch capsule, or a cellulosic capsule. Although not limited to capsules, such dosage forms can further be coated with, for example, a seal coating, an enteric coating, an extended release coating, or a targeted delayed release coating. These various coatings are known in the art, but for clarity, the following brief descriptions are provided: seal coating, or coating with isolation layers: Thin layers of up to 20 microns in thickness can be applied for variety of reasons, including for particle porosity reduction, to reduce dust, for chemical protection, to mask taste, to reduce odor, to minimize gastrointestinal irritation, etc. The isolating effect is proportional to the thickness of the coating. Water soluble cellulose ethers are preferred for this application. HPMC and ethyl cellulose in combination, or EUDRAGIT® E100, may be particularly suitable for taste masking applications. Traditional enteric coating materials listed elsewhere can also be applied to form an isolating layer.

Extended or delayed release coatings are designed to effect delivery over an extended period of time. The extended or delayed release coating is a pH-independent coating formed of, for example, ethyl cellulose, hydroxypropyl cellulose, methylcellulose, hydroxymethyl cellulose, hydroxyethyl cellulose, acrylic esters, or sodium carboxymethyl cellulose. Various extended or delayed release dosage forms can be readily designed by one skilled in art to achieve delivery to both the small and large intestines, to only the small intestine, or to only the large intestine, depending upon the choice of coating materials and/or coating thickness.

Enteric coatings are mixtures of acceptable excipients which are applied to, combined with, mixed with or otherwise added to the carrier or composition. The coating may be applied to a compressed or molded or extruded tablet, a gelatin capsule, and/or pellets, beads, granules or particles of the carrier or composition. The coating may be applied through an aqueous dispersion or after dissolving in appropriate solvent. Additional additives and their levels, and selection of a primary coating material or materials will depend on the following properties: 1. resistance to dissolution and disintegration in the stomach; 2. impermeability to gastric fluids and drug/carrier/enzyme while in the stomach; 3. ability to dissolve or disintegrate rapidly at the target intestine site; 4. physical and chemical stability during storage; 5. non-toxicity; 6. easy application as a coating (substrate friendly); and 7. economical practicality.

Dosage forms of the compositions of the present invention can also be formulated as enteric coated delayed release oral dosage forms, i.e., as an oral dosage form of a composition as described herein which utilizes an enteric coating to affect release in the lower gastrointestinal tract. The enteric coated dosage form may be a compressed or molded or extruded tablet/mold (coated or uncoated) containing granules, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

Delayed release generally refers to the delivery so that the release can be accomplished at some generally predictable location in the lower intestinal tract more distal to that which would have been accomplished if there had been no delayed release alterations. The preferred method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention to achieve delivery to the lower gastrointestinal tract. Polymers for use in the present invention are anionic carboxylic polymers.

Shellac, also called purified lac, a refined product obtained from the, resinous secretion of an insect. This coating dissolves in media of pH>7.

Colorants, detackifiers, surfactants, antifoaming agents, lubricants, stabilizers such as hydroxy propyl cellulose, acid/base may be added to the coatings besides plasticizers to solubilize or disperse the coating material, and to improve coating performance and the coated product.

In carrying out the method of the present invention, the combination of the invention may be administered to mammalian species, such as dogs, cats, humans, etc. and as such may be incorporated in a conventional systemic dosage form, such as a tablet, capsule, or elixir. The above dosage forms will also include the necessary carrier material, excipient, lubricant, buffer, antibacterial, bulking agent (such as mannitol), anti-oxidants (ascorbic acid of sodium bisulfate) or the like.

The dose administered may be carefully adjusted according to age, weight and condition of the patient, as well as the route of administration, dosage form and regimen and the desired result.

The compositions of the invention may be administered in the dosage forms in single or divided doses of one to four times daily, or may be administered multiple times per day. It may be advisable to start a patient on a low dose combination and work up gradually to a high dose combination.

Tablets of various sizes can be prepared, e.g., of about 2 to 2000 mg in total weight, containing one or both of the active ingredients, with the remainder being a physiologically acceptable carrier of other materials according to accepted practice. Gelatin capsules can be similarly formulated.

Liquid formulations can also be prepared by dissolving or suspending one or the combination of active substances in a conventional liquid vehicle acceptable for administration so as to provide the desired dosage in, for example, one to four teaspoonfuls.

Dosage forms can be administered to the patient on a regimen of, for example, one, two, three, four, five, six, or other multiple doses per day

In order to more finely regulate the dosage schedule, the active substances may be administered separately in individual dosage units at the same time or carefully coordinated times. The respective substances can be individually formulated in separate unit dosage forms in a manner similar to that described above.

In formulating the compositions, the active substances, in the amounts described above, may be compounded according to accepted practice with a physiologically acceptable vehicle, carrier, excipient, binder, preservative, stabilizer, flavor, etc., in the particular type of unit dosage form.

### Packaging/Treatment Kits

The present invention relates to a kit for conveniently and effectively carrying out the methods in accordance with the present invention. Such kits may be suited for the delivery of solid oral forms such as tablets or capsules. Such a kit may include a number of unit dosages. Such kits can include a means for containing the dosages oriented in the order of their intended use. An example of a means for containing the dosages in the order of their intended uses is a card. An example of such a kit is a “blister pack”. Blister packs are well known in the packaging industry and are widely used for packaging unit dosage forms. If desired, the blister can be in the form of a childproof blister, i.e. a blister that is difficult for a child to open, yet can be readily opened by an adult. If desired, a memory aid can be provided, for example in the form of numbers, letters, or other markings or with a calendar feature and/or calendar insert, designating the days and the sections of a day in the treatment schedule in which the dosages can be administered, such as, for example, an AM dose is packaged with a “midday” and a PM dose.; or an AM dose is packaged with a PM dose. Alternatively, placebo dosages, or vitamin or dietary supplements, either in a form similar to or distinct from the active dosages, can be included.

The invention provides compositions, including preparations, formulations and/or kits, comprising combinations of ingredients, as described above (including the multi-ingredient combinations of drugs of the invention), that are serviceable as therapies for treating, preventing or improving conditions, states and disease as provided in the invention. In one aspect, each member of the combination of ingredients is manufactured in a separate package, kit or container; or, all or a subset of the combinations of ingredients are manufactured in a separate package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package consists two or more separate compartments. This blister package is made up of two separate material elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, the invention provides for blister packs, clamshells or trays comprising a composition (e.g., a (the multi-ingredient combination of drugs of the invention) combination of active ingredients) of the invention. Blister packs, clamshells or trays can be designed to be non-reclosable, so consumers can tell if a package has already opened. They are used to package for sale goods where product tampering is a consideration, such as the agents of the invention. In one aspect, a blister pack of the invention comprises a moulded PVC base, with raised areas (the “blisters”) to contain the tablets, pills, etc. comprising the combinations of the invention, covered by a foil laminate. Tablets, pills, etc. are removed from the pack either by peeling the foil back or by pushing the blister to force the tablet to break the foil. In one aspect, a specialized form of a blister pack is a strip pack.

In one aspect, a blister pack also comprises a method of packaging where the compositions comprising combinations of ingredients of the invention are contained in-between a card and clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. of the invention are used, and they can comprise of two vacuum-formed PVC sheets meshed together at the edges, with the informative card inside.

In one aspect, blister packaging comprises at least two components (e.g., is a multi-ingredient combination of drugs of the invention): a thermoformed “blister” which houses the product (e.g., a combination of the invention), and then a “blister card” that is a printed card with an adhesive coating on the front surface. During the assembly process, the blister component, which is most commonly made out of PVC, is attached to the blister card using a blister machine. Conventional blister packs can also be sealed.

As discussed herein, the products of manufacture of the invention can comprise the packaging of the therapeutic drug combinations of the invention, alone or in combination, as “blister packages” or as a plurality of packettes, including as lidded blister packages, lidded blister or blister card or packets, or a shrink wrap.

In one aspect, any of the invention's products of manufacture, including kits or blister packs, include memory aids to help remind patients when and how to take the agents of the invention.

The treatment kits can be constructed in a variety of forms familiar to one of ordinary skill in the art. The kits comprise at least one unit dosage of an active for administration according to a daily regimen and a means for containing the unit dosages. The treatment kits can, for example, be constructed for administration once daily, twice daily, thrice daily, four times daily, multiple administrations daily, or other dosage regimens. The kits comprise a means for the daily administration of an agent of the invention. In one embodiment the kits include from about one to about four unit dosages.

In one embodiment, the means for containing the unit dosages is a card, including, for example, a card that is capable of being folded. This card will be referred to herein as a main card, or alternatively a principal card or a first card, to distinguish it from additional optional cards, circulars, or other such materials which can be associated with the kit. This main card can be folded with a simple crease, or alternatively, with a double crease, so as to exhibit a spine, similar to the spine of a closed book. The main card can comprise a printable surface, i.e. a surface upon which the product name, appropriate administration instructions, product information, drawings, logos, memory aids, calendar features, etc. can be printed. The main card can comprise a means for containing said unit dosage or different dosages designated for different time of the day, and a memory aid for administering said unit dosage or dosages. The main card, especially if it is prepared from two or more laminated paperboard surfaces, can comprise a slit or pocket, for example in one of the inner paperboard surfaces of the folded card. The slit or pocket can be used to contain a removable secondary card, i.e. a second card or insert card, which is not permanently attached or affixed to the main card.

The memory aid can include a listing of the days of the week, i.e. Sunday, Monday, Tuesday, Wednesday, Thursday, Friday, and Saturday, with appropriate spaces for the patient to select and indicate on the card the preferred day of the week on which to administer the therapy. The memory aid can include a listing of the time of day with appropriate spaces for the patient to select and indicate on the card the preferred time of day (e.g.: AM, PM, midday) at which to administer the therapy. The memory aid can also include removable stickers having an appropriate pressure sensitive adhesive to facilitate easy removal and refastening to a desired surface such as a calendar or dayminder. The removable stickers can be located on the main card, or can be located on the secondary card which is constructed so that it can be readily inserted into and removed from the optional slit in the main card. Additionally, the optional slit can contain additional patient information and other circulars.

Other means for containing said unit dosages can include bottles and vials, wherein the bottle or vial comprises a memory aid, such as a printed label for administering said unit dosage or dosages. The label can also contain removable reminder stickers for placement on a calendar or dayminder to further help the patient to remember when to take a dosage or when a dosage has been taken.

The invention will be illustrated in more detail with reference to the following Examples, but it should be understood that the present invention is not deemed to be limited thereto.

### EXAMPLES

### Example 1

### Product Formulation

The preferred form for the targeted delivery of probiotic microorganisms is capsule which offers most protection for its live content during the formulation process as compared to other pharmaceutical forms. It is specialty coated to achieve delayed targeted release. The coating method in this example is pH dependent that is most economical, easy to apply and offers flexibility as to the desired release characteristics.

I. Vcaps (Capsugel) made of HPMC material are selected for the delayed release probiotic formulation. Vcaps brand has an advantage of easy coating due to matt surface of the capsules. Different coating is used for the distal small intestinal and colonic delivery.

i) For distal small intestinal delivery the material used for coating is EUDRAGIT® L30 D-55 (Evonic Industries, Darmstadt, Germany) that dissolves at pH of 5.5-6.0. The mean time for complete disintegration of capsules coated with Eudragit L30 D-55 was 2.4 hours in the in vivo tests (18). An alternative coating can be done utilizing Eudragit L30 D-50 which has similar dissolution characteristics (U.S. Pat. No. 7,094,425 B2). The preferred thickness of the coating is 8 mg/cm2, although further adjustments to increase the time to complete disintegration of capsules can be made by increasing the thickness of coating up to 15 mg/cm2.

ii) For colonic delivery, coating with EUDRAGIT® FS 30D (Evonic Industries, Darmstadt, Germany) can be used that dissolves at pH of 7.0 and higher. The in vivo tests showed complete disintegration of capsules with thickness of the coating of 6 mg/cm2 at mean 6.9 hours (18). Similar results were reported for the in vitro testing (U.S. Pat. No. 7,094,425 B2).

a) An alternative coating method for colonic delivery utilizes 3:1 mixture of EUDRAGIT® L100 and Eudragit S100 (U.S. Pat. No. 6,228,396 B 1). Starch capsules such coated were shown in the in vivo testing to disintegrate primarily in the colon with mean time to dissolution of 6.0 hours.

b) An alternative coating method is to create separately the inner and the outer coat using EUDRAGIT® polymers (U.S. Pat. No. 5,686,105).

Application of coating for either distal small bowel or colonic delivery has its disadvantage of exposure of the formulated material to humidity and elevated temperature of up to 35° C. during coating application and up to 40° C. for 2 h during capsule drying (data from

Evonic Industries, Darmstadt, Germany). Such exposure may have an effect on the viability of the probiotic content of capsules. To prevent this, Vcaps can be coated prior to filling (20). The pre-coated capsules show good stability and are particularly suitable for probiotic use.

II. Probiotic microorganisms are formulated as suspension in a prefilled syringe for administration through a catheter directly into the intestine. Larger quantity of microorganism can be used, up to 1×10¹² cells per administration. The preferred method of preparation of the microorganisms is the oil stabilized stock probiotic microorganism (U.S. Pat. No. 8,114,397 B2) or using microencapsulation of probiotic microorganisms (US Patent Application US 2012/0039998 A1 and US 2005/0266069A1) prior to creating suspension that will be used for filling of the syringe. Pre-formulated syringe is stored protected from light exposure and in ambient room temperature, or refrigerated at +4° C. After administration when the syringe is emptied, there is a “chase” saline used to flush the catheter. If the catheter is not of an indwelling type it will have to be safely removed and disposed using precautions for handling of infective material.

### Example 2

### Probiotic Content of the Delayed Release Formulation

Probiotic microorganisms in this invention can be anaerobic and aerobic bacteria, yeast and other probiotic microorganisms. The formulation may also contain other substances known to have beneficial effects on the functions of the gastrointestinal tract such as mannanoligosaccharide and contain substances required for specific formulation such as excipients, fillers, binders, plasticizer, etc. The microorganisms can be prepared as powder by lyophilization, vacuum drying, heat drying and other. The improved viability and stability in storage may be achieved by using oil based formulation (U.S. Pat. No. 8,114,397 B2) or using microencapsulation of probiotic microorganisms prior to filling the formulation (US Patent Application US 2012/0039998 A1 and US 2005/0266069A1).

The formulated material is to be stored in containers non-transmitting light and using desiccants pouches to eliminate humidity. If vegetative anaerobic organisms are used, they need to be stored prior to formulation and formulated and packaged without exposure to air oxygen. The preferred packaging method is the aluminum blister pack.

i) The capsules contains Bacteroides as a single species or combination of species: B. ovatus, B. vulgarus, B. thetaiotaomicron; 5-25 billion cells per capsule

ii) The capsules contains Clostridium species as a single species or combination of species in their vegetative or spore form: C. bifermentans, C. difficile (non-toxin producing); 5-25 billion cells per capsule

iii) The capsule contains Lactobacillus species as a single species or combination of species: L. rhamnosus, L. fermentum, L. reuteri, L. acidophilus, L. bulgaricus, L. crispatus, L. casei, L. paracasei, L. brevis, L. johnsoni, L. bifidum, L. delbrueckii; 5-25 billion cells per capsule.

iv) The capsule contains Bifidobacterium as a single species or combination of species: B. lactis, B. breve, B. longum, B. infantis, B. bifidum, B. adolescentis, B. thermophilum; 5-25 billion cells per capsule.

v) The capsule contains combination of probiotic species 5-25 billion cells per capsule.

vi) The capsule contains other microorganism species as a single species or combination of species that may include other probiotic bacteria: Escherichia coli 1917 (Nissle), Streptococcus thermophilus, Faecalibacterium prausnitzii, Butyricoccus pullicaecorum; 5-25 billion cells per capsule.

vii)The capsules contains lyophilized yeast Saccharomyces boulardii; 5 to 20 billion cells per capsule.

While the invention has been described in detail and with reference to specific examples thereof, it will be apparent to one skilled in the art that various changes and modifications can be made therein without departing from the spirit and scope thereof.

### REFERENCES

1. Quorum sensing: cell to cell communication in bacteria. Walters C M, Bassler B L Ann Rev Cell Dev Biol (2006) 21:319-346
2. Quorum sensing in Clostridium difficile: analysis of a lux S type signaling system. Carter G P, et al. Medical Microbiol (2005) 54, 119-127
3. Fecal Flora Reconstitution for recurrent Clostridium difficile infection: Results and Methodology. Rolke F., Syrawicz CM, Stoliman N. J. Clin. Gastroenterology (2010) 44, 567-570
4. Endoscopic fecal microbiota transplantation “First Line” treatment for severe Clostridium difficile infection? Brandt L J, Borody T J, Campbell J C J. Clin. Gastroenterology 2011, 45, 655-657
5. Review article: faecal transplantation therapy for gastrointestinal disease. Landy J, Al-Hassi H O, Walker A W, Cicilitira P J, et al. Alimentary Pharmacology and Therapeutics (2011) 34, 409-4015
6. Saccharomyces boulardii in the prevention of antibiotic associated diarrhea in children: A randomized double-blind placebo-controlled trial. Kotowska M, Albrecht P, Szajewska H. Aliment. Pharmacol Therap. 21:583-590
7. Systematic review and metaanalysis of Saccharomyces boulardi in adult patients. McFarland L V. World J. Gastroent. 16: 439-448
8. Bartlett J G: Clostridium difficile: history of its role as the enteric pathogen and the current state of knowledge about the organism. Clin. Inf. Diss. 1994, 18 (suppl 4): 5265-5272
9. Rea M C, O'Sullivan O, Shanahan F.: Clostridium difficile carriage in elderly subjects and associated changes in the intestinal microbiota. J. Clin. Microbiology 50: 867-875, 2012
10. Khoruts A, Dickveds J, Jansson J, Sadowsky M J: Changes in the composition of the human fecal microbiome after bacteriotherapy for recurrent clostridium difficile-associated diarrhoea. J. Clin Gastroenterol. 44:354-60, 2010
11. Gough E, Shaikh H, Manges, A R: Systematic review of intestinal microbiota transplantation (Fecal Bacteriotherapy) for recurrent Clostridium difficile infection. Clinical Infectious Diseases 53:994-1002, 2011
12. Landy L, Al-Hassi H O, Walker A W, et al.: Review article: faecal transplantation therapy for gastrointestinal disease. Alim. Pharm. & Therap. 34:409-415, 2011
13. Guo B, Louie T, Veldhuyzen van Zanten S, Dieleman L A: Systematic review: faecal transplantation for treatment of Clostridium difficile-associates disease. Alim. Pharm. & Therap. 35: 865-875, 2012
14. Tvede M, Rask-Madsen J: Bacteriotherapy for chronic relapsing Clostridium difficile diarrhoea in six patients. Lancet May 27, 1989:1156-1160
15. Maroo S, Lamont J T: Recurrent Clostridium difficile. Gastroenterology 130:1311-1316, 2006
16. Brandt L J, Reddy S S: Fecal microbiota transplantation for recurrent Clostridium difficile infection. J Clin. Gastroenterol. 45 Supp. 3 S159-S167, 2011
17. Balamuralidhara V, Pramodkumar T M, Srujana N, et al.: pH sensitive drug delivery systems: A review. American Journal of Drug Discovery and Development 1 (1) 24-48, 2011
18. Cole T E, Scott R A, Connor A L, et al.: Enteric coated capsules designed to achieve intestinal targeting. Intl. Journal of Pharmaceutics 231, 83-95, 2002
19. Watts P, Smith A.: Targit technology: coated starch capsules for site specific drug delivery into the lower gastrointestinal tract. Expert Opinion Drug Delivery 1:159-167, 2005,
20. Huyghebaert N, Vermeire A, Remon J P: Alternative method for enteric coating of HPMC capsule resulting in ready-to-use enteric-coated capsules. European Journal of Pharmaceutical Sciences 21:617-623, 2004
21. Niwa K, Takaya T, Morimoto T, Takada K: Preparation and evaluation of time controlled release capsule made of ethylcellulose for colon delivery of drugs. J. Drug Target. 3:83-89, 1995
22. Jeong Y I, Ohno T, Hu Z, Yoshikawa Yet al.: Evaluation of an intestinal pressure-controlled colon delivery capsule prepared by a dipping method. J. Controlled Rel. 71:175-182, 2001
23. Pozzi F, Furlani P, Gazzaniga A, et al: The TIMECLOCK system: a new oral dosage form for fast and complete release of drug after a predetermined lag time. J. Controlled Release. 31:99-108, 1994
24. Ueda S, Ibuki R, Kawamuza A, et al: Development of a novel drug delivery system , Time-Controlled-Explosion system [TES]. Drug Targeting 2:133-140,1994
25. Gupta V K, Assmus M W, Beckert T E, Price J C: A novel pH and time-based multi-unit potential colonic drug delivery system. II Optimization of multiple purpose variables. Intl. J. Pharma. 213:93-102, 2001
26. Scheline R R: Metabolism of foreign compounds by gastrointestinal microorganisms. Pharmacol. Rev. 25:451-523, 1973
27. Van Den Mooter G, Samyn C, Kinget R: Azo polymers for colon specific drug delivery. Intl. J. Pharm. 87:37-46, 1994
28. Schacht E, Gevaert A, Kenawy E R, et al.: Polymers for colon specific drug delivery. J Controlled Rel. 39:327-338, 1996
29. Terkoglu M, Ugarlu T.: In vitro evaluation of pectin-HPMC compression coated 5-aminosalicylic acid tablets for colonic delivery. Eur. J. Pharm. Sci. 53:65-73, 2002
30. Fetzner A, Bohm S, Schreder S, Schubert R: Degradation of raw or film incorporated β-cyclodextrin by enzymes and colonic bacteria. Eur. J. Pharm. Sci. 58:91-97, 2004
31. Macleod G S, Fell J T, Collet J H, et al.: Selective drug delivery to the colon using pectin hydroxypropyl methylcellulose film coated tablets. Intl. J. Pharm. 1897:251-257, 1999

1. A method of treatment or prevention of Clostridium difficile infection in a patient in need of such treatment or prevention by administration of a probiotic delivery system for administering a plurality of live probiotic microorganisms to the intestine of an individual in need thereof, said probiotic delivery system comprising:
a probiotic agent, wherein said probiotic agent comprises at least one species of live probiotic microorganisms;
optionally, at least one additional agent selected from the group consisting of antibiotics, bismuth containing compounds, intestinal motility agents, and combinations thereof; and
a delivery vehicle,
wherein the delivery vehicle will pass intact through the stomach, the duodenum and the upper small intestine to release the probiotic agent in the distal ileum and/or the colon of the intestine of the individual, further wherein the probiotic agent is capable of suppressing growth of C. diff.

2. The method of claim 1, wherein the treatment can be used as an adjunct to antibiotic treatment of Clostridium difficile and treatment of antibiotic associated diarrhea.

3. The method of claim 1, wherein the treatment is prophylaxis to prevent initial and recurrent or refractory Clostridium difficile infection.

4. The method of claim 1, wherein the treatment is prophylactic use applied in patients at risk of Clostridium difficile infection that include antibiotic use, advanced age, co-morbidities known to predispose to Clostridium difficile infection, underlying disease severity, prolonged hospitalization, and exposure to other patients with Clostridium difficile infection.

5. The method of treating a disease or condition in a patient by using a personalized probiotic delivery system according to claim 1, wherein the means for containing said therapeutic dosage or dosages is selected from the group consisting of: the first and optional unit dosages are packaged together in a single package or packette; the first and optional unit dosages are packaged separately in a plurality of packages or packettes; a blister packet; a lidded blister; or blister card or packets; a shrink wrap, and with both drugs released upon opening of the single package or packette; a plurality of packages or packettes; blister packet; lidded blister or blister card or packets; or shrink wrap; a blister pack; a container; a vial; and a device.

6. The method of treating a disease or condition in a patient by using a personalized probiotic delivery system according to claim 1, wherein said system is selected from the group consisting of a blister package comprising: a) a rupturable substrate b) a layer forming one or more blisters over the rupturable substrate, wherein each of the one or more blisters contain one or more unit dosage forms.

7-13. (canceled)

14. An oral probiotic delivery system for administering live probiotic microorganisms to an individual in need thereof, said probiotic delivery system comprising:
a probiotic agent, wherein said probiotic agent comprises at least one species of live probiotic microorganisms;
optionally, at least one additional agent selected from the group consisting of antibiotics, bismuth containing compounds, intestinal motility agents, and combinations thereof; and
a delivery vehicle,
wherein the delivery vehicle will pass intact through the stomach, the duodenum and the upper small intestine to release the probiotic agent in the distal ileum and/or the colon of the intestine of the individual, further wherein the probiotic agent is capable of suppressing growth of C. diff.

15. The probiotic delivery system of claim 14, wherein the delivery vehicle is selected from the group consisting of pills, tablets, caplets, capsules, soft gels, and coated probiotic granules, that will release the probiotic agent in the distal ileum and/or colon.

16. The probiotic delivery system of claim 14, wherein the additional agent is present, and is in a dosage form selected from immediate release, delayed release, extended release which is released in the distal ileum and/or colon, and targeted release which is targeted to be released in the distal ileum and/or colon.

17. The probiotic delivery system of claim 16, wherein the additional agent is targeted to release the additional agent in the distal ileum and/or colon of the intestine of the individual.

18. The probiotic delivery system of claim 15, wherein the coated probiotic granules are in a sachet.

19. The probiotic delivery system of claim 15, wherein the coated granules are selected from the group consisting of probiotic granules with an immediate release coating, probiotic granules with a delayed release coating, microencapsulated probiotic granules, and combinations thereof.

20. The probiotic delivery system of claim 19, wherein the coated granules are mixed as a suspension and administered orally.

21. The probiotic delivery system of claim 19, wherein the coated granules can be applied directly to food as a sprinkle.

22. (canceled)

23. The probiotic delivery system of claim 19, wherein the coated probiotic granules are in a capsule, which optionally has a coating selected from immediate release, delayed release, extended release which is released in the distal ileum and/or colon, and targeted release which is targeted to be released in the distal ileum and/or colon.

24. The probiotic delivery system of claim 19, wherein the coated probiotic granules are in a tablet or caplet, which optionally has a coating selected from immediate release, delayed release, extended release which is released in the distal ileum and/or colon, and targeted release which is targeted to be released in the distal ileum and/or colon.

25. The probiotic delivery system of claim 14, wherein the probiotic agent is primarily anaerobic bacteria, wherein the anaerobic bacteria is in vegetative, spore, or a mixture of vegetative and spore forms, further wherein the probiotic agent will promote development of healthy colonic bacterial flora .

26. (canceled)

27. The probiotic delivery system of claim 14, wherein the probiotic agent is primarily aerobic bacteria, wherein the aerobic bacteria is in vegetative, spore, or a mixture of vegetative and spore forms, further wherein the probiotic agent will promote development of healthy colonic bacterial flora.

28. The probiotic delivery system of claim 27, wherein the delivery vehicle protects the viability of the probiotic agent during storage and until their release in the distal ileum and/or colon.

29. The probiotic delivery system of claim 14, wherein the delivery vehicle is coated such that the probiotic agent is released in the distal ileum and/or colon.

30. (canceled)

31. The probiotic delivery system of claim 14, wherein the delivery vehicle is coated such that will ensure the delayed release of its content and will offer required shelf life stability.

32. The probiotic delivery system of claim 14, wherein the delivery vehicle is a capsule, wherein the capsule comprises materials selected from the group consisting of hydroxy-propyl-methyl-cellulose, gelatin, starch, and combinations thereof.

33. The probiotic delivery system of claim 14, wherein the delivery vehicle has a pH dependent coating.

34. The probiotic delivery system of claim 14, wherein the delivery vehicle is coated with a copolymer selected form the group consisting of methacrylic acid, methacrylates, and combinations thereof that dissolve at pH 5.5 to 7.0, and may be used to achieve distal ileum and/or colonic delivery.

35. The probiotic delivery system of claim 34, wherein the thickness of the coating constitutes additional factor that can be employed to adjust time required for disintegration of the coated formulation delivery vehicle.

36. The probiotic delivery system of claim 14, wherein the delivery vehicle is pre-coated prior to filling with the probiotic agent to reduce damaging effect of the formulation process on the probiotic agent, and will ensure tight and stable closure of delivery vehicle.

37. The probiotic delivery system of claim 14, wherein the delivery vehicle achieves distal small intestinal and/or colonic delivery by dissolution that is time dependent.

38. The probiotic delivery system of claim 14, wherein the delivery vehicle has delayed delivery using hydrogel plug.

39. The probiotic delivery system of claim 14, wherein the delivery vehicle time dependent delivery systems utilize ethylcellulose as the release determining polymer.

40. The probiotic delivery system of claim 14, wherein the delivery vehicle has delayed delivery based on a combination of pH dependent and time dependent systems.

41. The probiotic delivery system of claim 14, wherein the delivery vehicle has delayed delivery based on bacterial degradation of the formulation coating, such as degradation of polymers containing azo bonds that are cleaved by bacterial azo reductase enzyme.

42. The probiotic delivery system of claim 14, wherein the probiotic agent is selected from the group consisting of Streptococcus, Enterococcus, Staphylococcus, Micrococcus, Leuconostoc, Pediococcus, Stomatococcus, Corynebacterium, Arthrobacter, Brevibacterium, Rothia, Arcanobacterium, Aureobacterium, Microbacterium, Gardnerella, Kurthia, Bacillus, Escherichia, Enterobacter, Ewingella, Hafnia, Klebsiella, Kluyvera, Leciercia, Leminorella, Moellerella, Obesumbacterium, Pragia, Pantoea, Photorhabdus, Proteus, Providencia, Rahnella, Serratia, Tatumella, Citrobacter, Clostridium, Peptostreptococcus, Propionibacterium, Lactobacillus, Eubacterium, Bifidobacterium, Mobiluncus, Bacteroides, Porphyromonas, Prevotella, Fusobacterium, Sutterella, Bilophila, Butyrrivibrio, Catonella, Dialister, Johnsonella, Saccharomyces, Pichia, Faecalibacterium, Butyricoccus, and combinations thereof.

43. (canceled)

44. The probiotic delivery system of claim 42, wherein the probiotic agent is in vegetative or spore form.

45. The probiotic delivery system of claim 42, wherein the probiotic agent is healthy colonic bacterial flora cultured from donors selected from the patient, patient relatives, and unrelated human donors.

46-59. (canceled)

60. The method of claim 1 wherein the patient is a mammal.

61. The method of claim 1 wherein the patient is human.

62. The method of claim 5 wherein the patient is a mammal.

63. The method of claim 5 wherein the patient is human.

64-65. (canceled)

66. The oral probiotic delivery system of claim 14 wherein the individual is a mammal.

67. The oral probiotic delivery system of claim 14 wherein the individual is a human.

68-69. (canceled)
